(19) United States
(12) Patent Application Publication
(10) Pub. No.: US 2026/0057975 A1
(43) Pub. Date: Feb. 26, 2026

(54) ARTIFICIAL INTELLIGENCE APPLICATIONS TO ALERT A DATA SAFETY MONITORY BOARD TO ADVERSE EVENTS IN CLINICAL-TRIAL DATA

(71) Applicant: Telperian, Inc., Austin, TX (US)

(72) Inventors: Kyle McArthur, Austin, TX (US); Ryan Hafen, Austin, TX (US); Brian Hobbs, Austin, TX (US); Rick Landin, Austin, TX (US)

(21) Appl. No.: 19/306,920

(22) Filed: Aug. 21, 2025

Related U.S. Application Data
(60) Provisional application No. 63/685,579, filed on Aug. 21, 2024.

(51) Int. Cl. G16H 10/20

(52) U.S. Cl. CPC G16H 10/20

(57) ABSTRACT

Provided is a process including: accessing clinical trial data of an ongoing clinical trial that is not yet complete, the clinical trial having a plurality of treatment groups and a plurality of patients in the treatment groups; detecting, with an anomaly detection model, an anomaly in the clinical trial data for a first patient, the anomaly corresponding to a first patient among the plurality of patients; in response to detecting the anomaly classifying the first patient as anomalous in the trial; generating, with the computer system, by an artificial intelligence model, a candidate explanation for the detected anomaly by designating data in a record for the first patient as potentially correlated with the anomaly; and causing the anomaly and the candidate explanation to be presented to a data safety monitoring board (“DSMB”) of the clinical trial.

### CROSS-REFERENCE TO RELATED APPLICATIONS

This patent claims the benefit of U.S. Provisional Patent Application 63/685,579, titled ARTIFICIAL INTELLIGENCE APPLICATIONS TO ALERT A DATA SAFETY MONITORY BOARD TO ADVERSE EVENTS IN CLINICAL-TRIAL DATA, filed 21 Aug. 2024. The entire content of each afore-mentioned patent filing is hereby incorporated by reference.

### BACKGROUND

### 1. Field

The present disclosure relates generally to artificial intelligence, and more specifically to artificial intelligence (AI) applications to alert a data safety monitoring board (“DSMB”) to adverse events in clinical trial data.

### 2. Description of the Related Art

Existing computer systems are not well suited for computational challenges that arise in the context of a DSMB seeking to analyze time-series data about potentially heterogenous treatments and populations. To understanding these computational challenges, it helps to be familiar with the operation of a DSMB, but first it should be noted that discussion of a DSMB should not be read to imply that the present application is directed to the human interactions or mental processes that unfold in the context of a DSMB, rather than the specific technical challenges in developing computer systems that are useful in this context.

In clinical trials, data is gathered and organized systematically to monitor the safety of participants throughout the study. This process involves several steps to ensure that any potential safety concerns can be identified and addressed promptly before the trial is complete.

Often data collection begins with the enrollment of participants. Each participant's health status is typically documented, including baseline measurements such as vital signs, lab results, and medical history. This baseline data may serve as a reference point for comparing any changes that occur during the trial.

As the trial progresses, data is often repeatedly collected from participants, e.g., at scheduled intervals. This data may include information about any side effects, adverse events, and changes in health status that occur during the study. Participants might undergo regular physical examinations, laboratory tests, and other assessments depending on the nature of the trial. These data points may be recorded in case report forms (CRFs), or other documents designed to capture all relevant information, e.g., in a consistent manner.

The data collected may be entered into a database, where it is cleaned and organized. Data cleaning often involves checking for errors, inconsistencies, or missing information, checking that the dataset is accurate and complete. The organized data is typically categorized by various factors, such as the type of adverse event, severity, and time of occurrence. This categorization helps in analyzing patterns and trends within the data.

To identify signals relevant to safety, often the DSMB repeatedly (e.g., periodically during the trial) reviews the data as it accumulates. A DSMB is typically an independent group of experts responsible for overseeing patient safety and the effectiveness of treatments during a clinical trial. The DSMB's typical role is to safeguard the well-being of participants and ensure the data collected throughout the trial is accurate and reliable. This group often regularly reviews the data as it accumulates, looking for any signs of potential safety issues. If the DSMB detects adverse events or side effects that could harm participants, it in many cases has the authority to recommend changes to the trial's protocol or even stop the trial if necessary to protect those involved. In addition to monitoring safety, the DSMB often evaluates whether the trial is achieving its intended outcomes. If early results indicate that the treatment being tested is clearly effective or ineffective, the DSMB might suggest ending the trial ahead of schedule to avoid unnecessary risks or to bring a beneficial treatment to market more quickly. The board also often ensures that the trial is conducted ethically and that the data is collected without bias. This includes making sure that all participants are treated according to the established protocol and that there is no misconduct during the trial. Members of a DSMB typically include clinicians, biostatisticians, ethicists, and other relevant experts. These individuals are independent of the study sponsors and investigators, which helps prevent conflicts of interest and ensures that decisions are made objectively.

The DSMB review process often involves statistical analyses to detect any unusual patterns or trends that might indicate a safety concern. For example, if a particular side effect is reported more frequently than expected, this could be a signal that warrants further investigation. The data is often compared against predefined safety thresholds or benchmarks, which are established based on prior knowledge from earlier phases of the trial or similar studies.

Interim analyses are also often conducted at specific points during the trial. These analyses are often planned in advance and involve a comprehensive review of the safety data collected up to that point. The DSMB typically performs these interim analyses to assess whether the trial should continue, be modified, or be stopped early due to safety concerns.

### SUMMARY

The following is a non-exhaustive listing of some aspects of the present techniques. These and other aspects are described in the following disclosure.

Some aspects include applying AI to clinical-trial safety data to alert the DSMB to adverse events, which may be related to efficacy outcomes, concomitant medications, prior medications, etc. Some embodiments also construct patient profiles based on these adverse events and present those profiles to members of the DSMB to inform treatment decisions for the specific patient, groups of patients, or for the overall study design.

Some aspects include a process, including: accessing, with a computer system, clinical trial data of an ongoing clinical trial that is not yet complete, the clinical trial having a plurality of treatment groups and a plurality of patients in the treatment groups; detecting, with the computer system, with an anomaly detection model, an anomaly in the clinical trial data for a first patient, the anomaly corresponding to a first patient among the plurality of patients; in response to detecting the anomaly, with the computer system, classifying the first patient as anomalous in the trial; generating, with the computer system, by an artificial intelligence model, a candidate explanation for the detected anomaly by designating data in a record for the first patient as potentially correlated with the anomaly; and causing, with the computer system, the anomaly and the candidate explanation to be presented to a data safety monitoring board (“DSMB”) of the clinical trial.

Some aspects include a tangible, non-transitory, machine-readable medium storing instructions that when executed by a data processing apparatus cause the data processing apparatus to perform operations including the above-mentioned process.

Some aspects include a system, including: one or more processors; and memory storing instructions that when executed by the processors cause the processors to effectuate operations of the above-mentioned process.

### BRIEF DESCRIPTION OF THE DRAWINGS

The above-mentioned aspects and other aspects of the present techniques will be better understood when the present application is read in view of the following figures in which like numbers indicate similar or identical elements:

FIG. 1 is a block diagram of an example DSMB workflow management system in accordance with some embodiments;

FIG. 2 is a flowchart of a DSMB workflow management process in accordance with some embodiments; and

FIG. 3 illustrates an example of a computing device by which the present techniques may be implemented, e.g., in a collection of such devices forming a computing system, communicating with one another via one or more networks.

While the present techniques are susceptible to various modifications and alternative forms, specific embodiments thereof are shown by way of example in the drawings and will herein be described in detail. The drawings may not be to scale. It should be understood, however, that the drawings and detailed description thereto are not intended to limit the present techniques to the particular form disclosed, but to the contrary, the intention is to cover all modifications, equivalents, and alternatives falling within the spirit and scope of the present techniques as defined by the appended claims.

### DETAILED DESCRIPTION OF CERTAIN EMBODIMENTS

To mitigate the problems described herein, the inventors had to both invent solutions and, in some cases just as importantly, recognize problems overlooked (or not yet foreseen) by others in the fields of statistics, artificial intelligence, and clinical-study design. Indeed, the inventors wish to emphasize the difficulty of recognizing those problems that are nascent and will become much more apparent in the future should trends in industry continue as the inventors expect. Further, because multiple problems are addressed, it should be understood that some embodiments are problem-specific, and not all embodiments address every problem with traditional systems described herein or provide every benefit described herein. That said, improvements that solve various permutations of these problems are described below.

Often a DSMB faces significant challenges in managing information related to clinical trials. In many cases, different members have different preferences for data formats, and they are often constrained by limitations of the formats in handling large volumes of data efficiently. Existing software tools are generally not designed to address the particular needs of the DSMB or address technical challenges that arise, like algorithmically constructing patient profiles with the information most relevant to triaging issues brought to the DSMB's attention, which can be challenging when patient data is relatively high dimensional and the information most relevant to such an issue might entail a relationship between a subset of those dimensions that is not self-evident. Brute force exploration of every permutation of such relationships may be too computationally resource intensive and slow in some cases. Existing software tools are also not well suited for detecting rare events, which are by their nature, difficult to train many AI models to detect in virtue of the sparsity of training examples and highly diverse set of potential events to be detected. Further, many existing software tools are not well-suited for identifying sub-populations for which a trial is working (or not working) or for whom risk-benefits tradeoffs are warranted (or are not), to guide decisions by a DSMB. Identifying such sub-populations is, again, another instance of what is often called the “curse of dimensionality” in patient data, where the number of permutations of sub-populates to be considered scales factorially with the number of dimensions of data gathered about patients. Brute force approaches are not suitable in many cases. None of this is to suggest that any of these approaches are disclaimed or disavowed (or that discussion of any other challenges or tradeoffs herein is a disclaimer or disavowal).

DSMB workflows are often impaired by the tools and data formats available. PDF files, while convenient for presenting data in a visually organized way, are generally static documents, lacking the flexibility needed for detailed analysis, especially when dealing with complex datasets like those in clinical trials. Older physicians may prefer PDFs because they are easy to read and resemble traditional paper reports. However, this preference can become a barrier when the task requires more than just reading the data—such as performing statistical analyses, filtering results, or combining datasets. PDFs are not easily manipulated; extracting data from them for further analysis often requires additional steps, such as manual entry or specialized software, which can introduce errors and consume time.

On the other hand, younger members of the DSMB may prefer CSV (Comma-Separated Values) files, which are much more flexible and suited to data analysis. CSV files can be easily imported into data analysis software, spreadsheets, or statistical tools, allowing for quick sorting, filtering, and manipulation of the data. However, the preference for CSV files can create friction if other members of the board are not comfortable using them. This divide can lead to inefficiencies, as the team may struggle to agree on a single format that meets everyone's needs, leading to delays in data review and analysis. Further, it can be hard to collaborate in relation to CSV files when every board member has a different format or analysis.

The problem is exacerbated by the sheer volume of data that clinical trials generate. Going through stacks of PDFs is not only time-consuming but also impractical, particularly when timely decisions are crucial for participant safety. This method of data delivery can lead to bottlenecks in the DSMB's workflow, as members may find it difficult to efficiently sift through large amounts of static data to identify safety signals or trends.

Some embodiments apply AI to clinical-trial safety data to alert the DSMB to adverse events, which may be related to efficacy outcomes, concomitant medications, prior medications, etc. Some embodiments also construct patient profiles based on these adverse events and present those profiles to members of the DSMB to inform treatment decisions for the specific patient, groups of patients, or for the overall study design. In some embodiments, this is done by leveraging the clinical data analysis platform and other techniques described in the following patent applications, the contents of which are hereby incorporated by reference:
a. U.S. Prov. Pat. App. 63/550,743|078525-0577457: SEARCHABLE AND NAVIGABLE ELECTRONIC SYSTEM FOR FINDING RESULTS FROM MANY ANALYSES APPLIED TO DATA;
b. U.S. Prov. Pat. App. 63/614,517|078525-0577453: REPRESENTING CLINICAL TRIAL DATA IN AN INTERACTIVE ANALYSIS PLATFORM;
c. U.S. Prov. Pat. App. 63/614,516|078525-0577455: DATA INTEGRITY FOR CLINICAL STUDIES; and
d. U.S. Prov. Pat. App. 63/567,392|078525-0579120: ENHANCING RETRIEVAL AUGMENTED GENERATION ACCURACY.

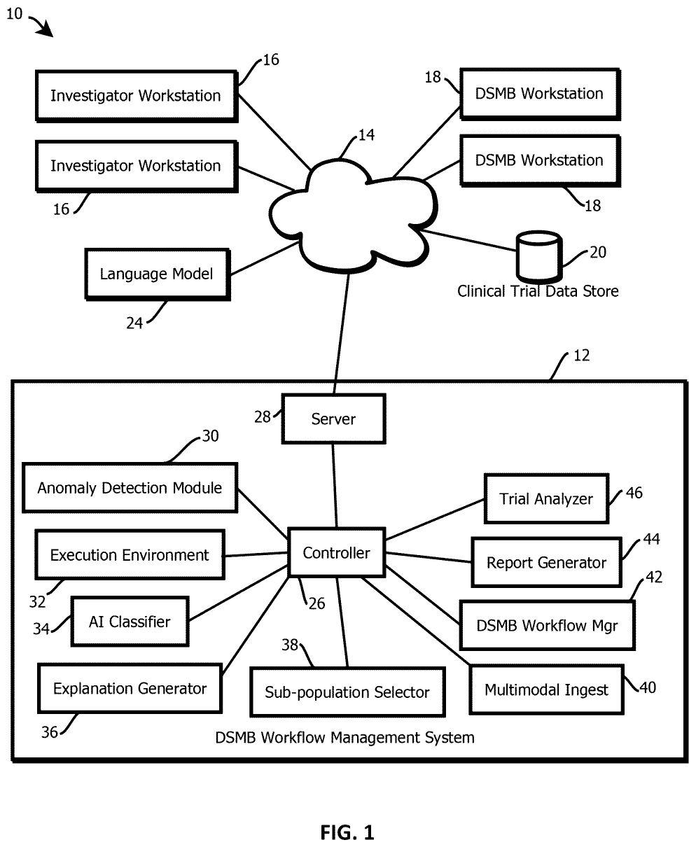

FIG. 1 is a block diagram of computing environment 10, which in some embodiments includes a DSMB workflow management system 12, the internet 14, investigator workstations 16, DSMB workstation 18, clinical trial data store 20, and language model 24. The DSMB workflow management system 12 in some embodiments includes controller 26, server 28, anomaly detection module 30, execution environment 32, AI classifier 34, explanation generator 36, sub-population selector 38, multimodal ingest module 40, DSMB workflow manager 42, report generator 44, and trial analyzer 46.

As described in greater detail below, the investigator workstations 16 may be desktop computers, laptop computers, tablet computers, wearable computers, kiosks, or have a variety of other formats. The same is true of the DSMB workstations 18. Two instances of each are illustrated, but embodiments are expected to include substantially more often geographically distributed computing devices. In some cases, these computing devices may interact with the system 12 through a web browser or a special purpose native application executing on the respective client computing device.

In some cases, clinical trial data to be analyzed for one or more clinical trials monitored by one or more DSMBs with the system 12 may be stored in one or more clinical trial data stores 20 which in some cases may be implemented with the techniques described in the patent applications incorporated by reference above. Tenant accounts and roles and permissions may be applied by the system 12 to isolate data and control access.

Some embodiments may interact with one or more language models 24 such as various foundation language models implemented with transformer architectures. Examples include those implementing multi-headed attention on decoder only models, and some embodiments may also interact with various computer vision models or multimodal transformer architectures that can also analyze images. In some cases, third-party optical character recognition models, speech-to-text models and other types of models may also be accessed via the internet 14, as described further below.

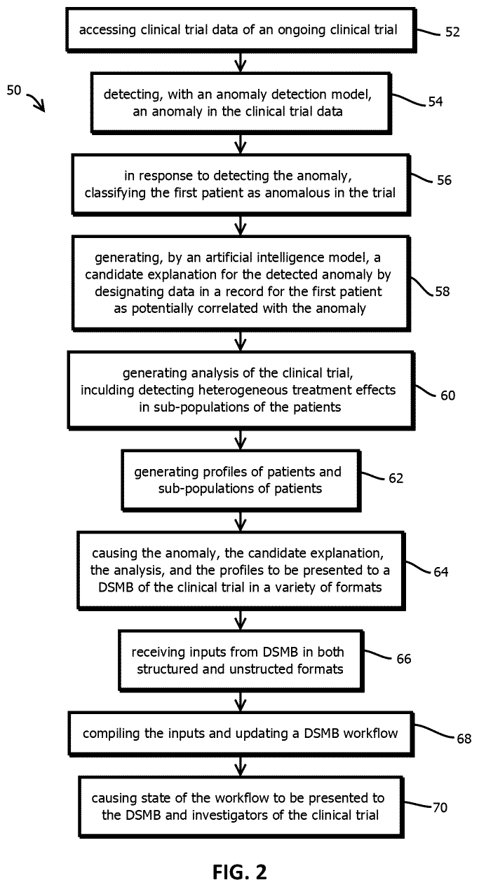

In some embodiments, the system 12 may perform operations described in greater detail below such as the operations of FIG. 2. In some embodiments the system 12 is implemented in a cloud computing environment remote from the other components illustrated. In some embodiments, the controller 26 may orchestrate the various operations and features of the system 12 as described in greater detail below.

In some embodiments the server 28 may be a non-blocking web server or API server that causes user interfaces on the various illustrated workstations 16 and 18 to be populated and receives input from those workstations. In some embodiments, the server 28 may also mediate interactions with the language model 24 and the datastore 20.

In some embodiments, the anomaly detection module 30 may execute one or more of the various anomaly detection algorithms described elsewhere herein, for example, to detect anomalous trending or outcomes for patients or subpopulations of patients.

In some embodiments, the execution environment 32 may execute executable code generated with the language model 24. Examples include the executable code described below generated in order to perform customized analyses on trial data or generate visualizations or summarizations as described below.

In some embodiments the AI classifier module 34 may execute one or more classification algorithms like those described below. In some cases, this may include classifying subpopulations of patients, classifying types of anomalies, and classifying trials as described in greater detail below.

In some embodiments, the explanation generator 36 may generate causal or non-causal explanations of anomalies, classifications, or heterogeneous treatment effects that are detected for subpopulations as described in greater detail below. Some embodiments may construct context using techniques like retrieval augmented generation in combination with the language model 24 to generate such explanations. Some embodiments may also be configured to construct visualizations as described in greater detail below to support those explanations.

In some embodiments the subpopulation selector 38 may select subpopulations for evaluation by the trial analyzer 46 as potentially having heterogeneous treatment effects that may warrant designating a treatment of the trial as suitable or not suitable for that subpopulation (e.g., harming or healing at greater than a threshold rate relative to a baseline with greater than a threshold confidence) but potentially reaching different results for other subpopulations.

In some embodiments, the multimodal ingest module 40 may be configured to receive feedback from the workstations 18 in both structured and unstructured format and convert the unstructured format into a structured data format that can be combined with other structured data input by DSMB members, as described in greater detail below.

In some embodiments, the DSMB workflow manager 42 may maintain a state of a DSMB for a clinical trial, which may include previous reports and outputs, previous inputs, analyses, meeting minutes, and the like to ascertain and track the state of a DSMB workflow. This may also include registering votes of members of the DSMB for various propositions (and determining whether propositions voted upon are passed), like stopping a trial for a subpopulation because a treatment is suitable or not suitable, or changing an endpoint of a clinical trial, for example. Other examples may include votes on changing a dosage or aspects of a treatment, in each case, as described further below.

In some embodiments, the report generator, 44, may be operative to output reports with analysis and information gathered and generated by the other illustrated components. In some cases, these outputs may be presented on the workstations, 18. In some cases, outputs may be presented in a variety of different formats, examples including printable document format (PDF) suitable for printing and markup by hand and structured tabular data like CSV files suitable for ingest into statistical analysis platforms as described in greater detail below.

Some embodiments may include a trial analyzer 46 operative to perform a variety of different analyses on clinical trial data described in greater detail below.

FIG. 2 is an example flowchart of a process 50 that may be performed by the system 12 described above or by other systems. Aspects of each of the steps in this process are elaborated on in the discussion that follows. In some embodiments, the process includes accessing clinical trial data of an ongoing clinical trial as illustrated by block 52. Embodiments may include detecting with an anomaly detection model an anomaly in the clinical trial data as indicated by block 54. Embodiments may, in response to detecting the anomaly, classify a first patient as anomalous in the trial as indicated by block 56. Some embodiments may generate by an artificial intelligence model a candidate explanation for the detected anomaly by designated data in a record for the first patient as potentially correlated with the anomaly, as indicated by block 58. Some embodiments may generate analysis of the clinical trial, including detecting heterogeneous treatment effects in subpopulations of the patients, as indicated by block 60. Some embodiments may generate profiles of patients and subpopulations of patients, as indicated by block 62. Some embodiments may cause the anomaly, the candidate explanation, the analysis, and the profiles to be presented to a DSMB of the clinical trial in a variety of formats, as indicated by block 64. Some embodiments may receive inputs from the DSMB in both structured and unstructured formats, as indicated by block 66. Some embodiments may compile the inputs and update a DSMB workflow, as indicated by block 68. Some embodiments may cause state of the workflow to be presented to the DSMB and investigators of the clinical trial, as indicated by block 70. As noted above, these steps and components have features described in greater detail in the sections that follow.

In some cases, the physical architecture of environment 10 may be distributed with computing devices communicating over a network, like the Internet, to form a computing system, or some embodiments may execute on a single computing device. In some cases, the architecture may be a SaaS architecture with a cloud-hosted server system. In some cases, the system may be a software as a service (SaaS) architecture with client computing devices interrogating data in, and supplying data to, the server via native applications or web browsers. The environment 10 may include a database, such as data store 20 (e.g., external or internal to system 12), in which the described clinical trial data is stored. Some embodiments may use 16 by which a clinical trial team member enters data, a clinical analysis data platform, and one or more computing devices, such as workstations 18, of DSMB members. In some embodiments, the clinical data analysis platform may provide a user interface, for instance, a web-based or native app-based user interface, to the computing devices, such as workstations 16 or 18, operated by those entering and viewing data. In some embodiments, the system 12 may execute the analyses and provide the visualizations described herein, for instance daily, in response to receiving new data, in response to receiving a request for such an analysis, or in response to a scheduled DSMB meeting.

### Trial Analysis Model Architecture

In some embodiments, a model's architecture in the system 12 may treat each patient as a multi-modal, longitudinal object comprising static covariates, time-varying measurements, treatment exposures, and event histories, where the draft identifies patient-level profiling with risk stratification, dose modification, compliance, efficacy and safety forecasting, and ethics of continuing as contemplated aspects. In some embodiments, inputs (e.g., from the data store 20, entered by an investigator) may include structured tables (e.g., demographics, concomitant medications, laboratory panels), timestamped administrations with dose amounts, adverse event logs with grades and outcomes, endpoint measurements, and derived features such as baseline-normalized deltas and visit-aligned rolling aggregates; outputs may include anchor-linked scores, recommended actions, and uncertainty intervals stored alongside dataset version identifiers to preserve provenance. In some embodiments, a representation stack may include: (a) an embedding map that may project each categorical field to a dense vector and each continuous field to a normalized scalar with missingness indicators; (b) a temporal encoder that may accept sequences of visit- or day-level feature vectors and may compute context-aware hidden states using gated temporal convolutions or transformer-style attention with time-gap encodings; and (c) a static pathway that may summarize baseline risk factors, where both temporal and static pathways may be fused through concatenation and a small multilayer perceptron to yield per-timepoint latent states. In some embodiments, training data may be partitioned by patient and visit windows to create input-output examples with left-truncated, right-censored horizons, and the model may be optimized jointly for multiple heads producing adverse event risks, efficacy probabilities, adherence states, and dose-response maps.

In some embodiments for patient-level risk stratification of adverse events, e.g., by the analyzer 46 or module 30, the system may compute near-term and horizon-based probabilities for one or more adverse event categories and grades conditioned on the patient's latent state and current treatment context, where the draft contemplates software that may identify adverse event patterns and support risk stratification. In some embodiments, inputs may include a sequence of measurements up to an index time t, recent dosing history with cumulative exposure features, and cohort-level priors. The transformation may apply a discrete-time hazard head that may output per-interval event probabilities with masking for post-event periods, or a cause-specific competing-risks head that may allocate probability mass across event types. The output may include a calibrated probability for each event type over a configurable horizon, along with visit-level attributions derived from sequence attention weights to record which measurements contributed to the score. In some embodiments, this may be described as: receive windowed sequence X[0:t] and treatment history A[0:t]; compute latent state hₜ via temporal encoder; compute hazard logits z=f(hₜ, A[t]); convert z to per-grade probabilities with a link function; apply isotonic or Platt-style calibration on validation folds; return calibrated risk vector and uncertainty from Monte Carlo dropout or deep ensembles. In some embodiments, anomaly detectors described elsewhere in the draft (e.g., isolation forests, one-class support vector machines, local outlier factor, autoencoders) may be run in parallel on feature deltas to flag off-manifold physiology, and flagged segments may be provided as auxiliary inputs to the hazard head to allow the network to condition on outlier states without forcing the supervised labels to absorb the anomaly signal.

In some embodiments for dose modification suggestions by the analyzer 46, a dose-policy head may map current latent state and planned dosing to a recommended discrete action chosen from {maintain, reduce by rule-defined step, hold, discontinue, re-challenge}, where the draft indicates that dose modification recommendations may be provided based on patient-specific factors. In some embodiments, inputs may include dose to date, exposure summaries, pharmacokinetic or pharmacodynamic surrogates when available, and adverse event risk outputs. The transformation may estimate a counterfactual outcome surface by evaluating predicted near-term safety and efficacy under candidate actions using g-computation with the temporal encoder rolled forward for K steps while clamping the action, with inverse-probability or augmented inverse-probability weights applied if treatment assignment may deviate from perfect randomization. Outputs may include an action with a justification record comprising predicted changes in risk and efficacy and a constraint audit indicating adherence to protocol guardrails such as maximum per-visit dose change. In some embodiments, this may be described as: for each action a in the permitted set, simulate forward K steps by unrolling the temporal model with action a and stochastic draws for measurement noise; aggregate predicted safety and efficacy summaries; select the action that may maximize a scalarized objective subject to hard constraints (e.g., “do not exceed Grade 3 risk probability threshold”), then return the recommended action and a vector of predicted deltas with confidence bounds computed from simulation variance.

In some embodiments configured to analyze data for compliance and adherence, the analyzer 46 may infer latent adherence states from pill counts, device ingestion sensors, infusion attendance, and ePRO or diary signals, where the draft contemplates compliance monitoring and forecasting. In some embodiments, inputs may include timestamped adherence indicators and gaps constructed from dispensing and return events. The transformation may use a hidden Markov model that may define states such as “high,” “partial,” and “low” adherence with emission distributions for observed counts and diaries, or a recurrent classifier that may estimate the probability of each state at time t using the temporal encoder. The output may include an estimated state trajectory and a next-visit adherence risk. In some embodiments, this may be described as: build observation vector oₜ with counts and self-reports; compute p(sₜ|o_{0:}_{ₜ}) with either forward-backward recursions (hidden Markov model) or softmax over f(hₜ); compute forecast p(sₜ₊₁|o_{0:}_{ₜ}) by one-step prediction; attach adherence-adjusted weights to downstream efficacy and safety forecasts by conditioning outcome heads on sₜ to reflect exposure dilution.

In some embodiments configured for efficacy forecasting, an efficacy head of analyzer 46 may produce patient-level near-term response probabilities or continuous endpoint projections conditioned on current treatment and adherence-adjusted exposure. Inputs may include prior response status, biomarker trajectories, imaging-derived summaries when available, and subgroup encodings derived from heterogeneous treatment effect learners. Transformations may include a generalized outcome head (e.g., logistic for binary response, ordinal for composite scales, Gaussian for continuous) with monotonic constraints with respect to beneficial biomarker directions. Outputs may include a predicted response probability or expected change by the next landmark and a cumulative efficacy probability curve out to a prespecified horizon. In some embodiments configured for time-to-event efficacy endpoints, the model may produce a visit-updated restricted mean survival time forecast by integrating discrete-time hazards produced by a survival head applied to the temporal latent state, with the output expressed as an expected time in the event-free state over a fixed window.

In some embodiments configured for safety forecasting, similar code in the analyzer 46 may drive continuous-value predictors for laboratory and vital sign trajectories and sequence-change detectors for thresholds, where the transformation may apply a residual forecaster that may take hₜ and last observation as input and may output a multi-step vector of expected values with error bands. In some cases, a parallel alarm head may compute the probability of crossing protocol-defined boundaries for each analyte within the next L intervals. In some embodiments, this may be described as: compute hₜ; for each laboratory variable v, compute forecast vector μₜ:t+L and variance σ²ₜ:t+L via a linear readout with learned noise; for each boundary rule r, compute probability of violation under a Gaussian or quantile forecast; return the set of violation probabilities and suggested monitoring intervals. In some embodiments, these safety forecasts may be fused with adverse event hazard outputs via a small calibrator network that may reconcile signal consistency across measurements and events.

In some embodiments, efficacy and safety trade-offs may be synthesized by a decision module of analyzer 46 that may evaluate a set of candidate actions (including “continue unchanged”) using the counterfactual rollouts described above and may compute, for each action, a scalar decision score formed by combining efficacy summaries and safety risks according to a study-specific decision policy containing tunable weights and hard constraints described in the charter. In some embodiments, the system may identify the Pareto set of actions that may not be strictly dominated on both efficacy and safety and may select among Pareto-feasible actions using pre-specified clinical thresholds rather than free-form optimization, producing a record that may list the action, the trade-off vector, and the anchors for the underlying evidence used in the computation. In some embodiments, a policy-check step may assess temporal consistency by recomputing the decision score across bootstrap resamples of visits and may write a stability field on the recommendation object that may describe the fraction of resamples producing the same action, which may be surfaced in the report UI but not treated as gating logic.

In some embodiments, patient-level profiling may assemble the outputs above into a single object per anchorable context (e.g., per patient and visit) containing, for example: latest adverse event risk stratification, dose modification recommendation and audit, adherence state and forecast, efficacy probability or expected change, safety forecast summaries, and the trade-off record, where the draft discloses that the platform may generate detailed patient profiles and narratives with visualizations. In some embodiments, the object may also store the posterior uncertainty per head, the model version, and the dataset version, and may expose pointers to conversation threads and evidence cards to maintain linkages across data and PDFs.

In some embodiments, a method for computing values relevant to ethics of continuing may be implemented by analyzer 46 an executed prior to, or during, interim or ad hoc reviews by the DSMB. In some embodiments, inputs may include the set of per-patient decision objects for the review cut, prespecified ethical criteria from the DSMB charter encoded as machine-checkable rules, subgroup definitions used for heterogeneous effect monitoring, and multiplicity controls for selective analyses. The computation may proceed in five stages. First, a rule-evaluation stage may apply deontic guardrails that may declare certain conditions as unacceptable regardless of expected efficacy (for example, probability of severe harm above a bound within any protected subgroup), emitting per-subgroup rule violations with anchors to source values. Second, a proportionality stage may compute, per subgroup, whether expected clinical benefit may exceed expected harm under continued therapy over a fixed horizon according to the decision module's trade-off vector and may attach a boolean field for each subgroup indicating whether proportionality may hold under worst-case parameter settings within prespecified sensitivity ranges. Third, a fairness consistency stage may test that no subgroup identified by protected attributes may receive a systematically higher harm-at-equal-benefit profile by evaluating disparities on standardized residuals of the trade-off score. When disparities may exceed a threshold, the stage may emit a remediation recommendation to adjust inclusion strategies or monitoring frequency rather than a treatment assignment change. Fourth, a respect-for-persons stage may verify that consent and withdrawal rates may not indicate coercion signals by comparing adherence state transitions and dropout patterns against historical priors, and may flag sites with anomalous patterns for site-level actions. Fifth, a recommendation synthesis stage may compose a trial-level proposal among continue, modify, pause, or stop and may enumerate required mitigations (e.g., dose cap, cohort exclusion, enhanced monitoring) with links to the subgroup rule violations and proportionality findings used in the reasoning record. In some embodiments, these steps may be consistent with the draft's description of documenting DSMB outputs and decisions on whether to continue or modify a study, and of supporting discussions around the ethical implications of continuing treatment for specific subgroups.

In some embodiments, heterogeneous treatment effect signals produced elsewhere in the system may flow into the patient-level and subgroup stages as covariates or gating conditions without constraining the primary architecture, and multiplicity and stability checks described for subgroup selection may be applied to ethics inputs as well to avoid selection bias in downstream recommendations. In some embodiments, the ethics method may store, for each recommendation, the model versions, data versions, calibration sets, and bootstrap identifiers used to compute supporting metrics so that later reconciliations may recover the exact computational context in which the recommendation was generated.

In some embodiments, study-, cohort-, and subgroup-level profiling by analyzer 46 (e.g., using classifier 34 to group) may aggregate patient-level outputs into design-facing quantities that include futility assessments, probability of success, and probability of superiority, where these analysis heads may be listed among contemplated aspects. In some embodiments, inputs may include interim snapshots of adjudicated outcomes, exposure histories, adherence summaries, and subgroup encodings derived from heterogeneous-treatment-effect learners, together with design metadata such as the primary estimand definition, analysis model family, decision thresholds, information-time schedule, and allocation ratios. Transformations may be applied in a hierarchical model that may borrow strength across sites and subgroups while preserving subgroup-resolved draws for decision calculations. Outputs may include posterior or sampling distributions for study-level effects and per-subgroup effects, predictive distributions for the final analysis metric under planned enrollment and follow-up, and anchor-linked decision records suitable for DSMB session use.

In some embodiments, a futility module of analyzer 46 may produce the probability that a study, cohort, or subgroup will not meet a pre-specified success criterion at the planned analysis, conditional on the data observed to date and on operational projections. Inputs may include the interim dataset Dₜ truncated at calendar time t, protocol decision criteria expressed as machine-checkable rules (for example, minimum response difference or hazard contrast thresholds), and a projection object that may describe expected future enrollment, loss to follow-up, administrative censoring, and visit schedules. The transformation may fit an outcome model family declared in the charter, for example a generalized outcome model for binary or continuous endpoints or a discrete-time survival head for time-to-event endpoints, and may return a posterior or sampling distribution for the effect parameter at time t. The transformation may then simulate many hypothetical future increments by sampling latent patient trajectories and outcomes consistent with Dₜ and with the projection object, refitting or updating sufficient statistics as required by the model family, and computing the final analysis metric for each simulated completion. The output may include the fraction of simulated completions that fail the success rule, reported as a futility probability with uncertainty bands from repeated simulation batches, along with a rationale object that may list the anchors to interim counts or effect estimates that most influenced the probability. This process may be applied at the study level, cohort level, or within subgroups surfaced elsewhere in the system.

In some embodiments, a probability-of-success model may compute the predictive probability that the planned final analysis will meet the protocol's success rule, conditioned on what has been observed to date. Inputs may mirror those of the futility head, with the success rule specified as a function that may accept the final analysis statistic, noninferiority or superiority margins when applicable, stratification factors, and multiplicity adjustments. The transformation may reuse the interim-fitted model and the projection object to generate draws of completed trials. For each draw, the pipeline may compute the analysis statistic under the chartered analysis plan, including stratified or covariate-adjusted estimators, and may record success if the rule evaluates to true. The output may include a scalar probability of success for the study and a vector of probabilities for each cohort or subgroup, optionally accompanied by sensitivity variants under alternative projections of adherence or event accrual. This model may be used to populate DSMB packets with study- and subgroup-level success probabilities that accompany patient-level narratives and risk assessments the platform may already produce.

In some embodiments, a probability-of-superiority model may compute the probability that a target arm may exceed a comparator by at least a prespecified margin at the analysis time. Inputs may include an estimand declaration that may specify the contrast (for example, difference in means for continuous outcomes, difference in response rates or odds ratios for binary outcomes, hazard or restricted-mean-survival-time contrasts for time-to-event outcomes), the margin, and covariate adjustment choices. The transformation may form a posterior or sampling distribution over the effect contrast from the interim data, optionally using a hierarchical layer that may admit subgroup-varying effects with partial pooling, and may then evaluate the probability mass above the margin. For designs that may require predictive assessment rather than current-evidence assessment, the model may further simulate future increments as described above and may compute the fraction of completed-trial draws whose estimated contrast exceeds the margin. The output may include probabilities of superiority for the overall study, each cohort, and each prespecified subgroup, with uncertainty intervals, and may store pointers to the interim effect estimates and calibration diagnostics used to inform the calculation. In some embodiments, survival endpoints may be handled by drawing discrete-time hazard trajectories from the interim-fitted survival head and converting them to contrasts in event probability or restricted mean survival time over a fixed window, before computing superiority probabilities.

In some embodiments, subgroup-level profiling for these quantities may reference subgroup definitions produced by heterogeneous treatment effect modules without constraining the analysis-model choice. Subgroup labels may be prespecified families from the charter or derived rules compressed from conditional-average-treatment-effect functions and then locked via discovery-confirmation splits before use in study-level decision heads, with multiplicity controls applied when many subgroups are evaluated. In some embodiments, the futility, probability-of-success, and probability-of-superiority heads may accept a list of subgroup filters and produce per-subgroup decision records, while also writing stability fields that may summarize bootstrap or cross-fold variability of each probability estimate.

In some embodiments involving interim governance, the outputs of the three heads or other models may be attached to structured decision criteria for DSMB use alongside qualitative discussions and safety profiling, and may be surfaced during sessions where continue, modify, pause, or stop proposals are recorded with anchors to supporting artifacts. The same study/cohort/subgroup profiles may be regenerated on a cadence aligned with DSMB meetings and may be documented in minutes that reflect the computed probabilities and the chartered thresholds used for decisions.

In some embodiments, the analyzer's 46 architecture may incorporate external evidence streams comprising synthetic data, real-world data from observational sources, and historical trial data, with these sources referenced among contemplated aspects of the draft. In some embodiments, a data harmonization module may ingest these sources, map fields onto study estimands and variable dictionaries, and persist provenance metadata including source identifier, collection window, coding system, and version tag, where the platform may further allow exploration of within-study data in relation to external datasets. The harmonization module may write a schema object that describes units, allowable ranges, category vocabularies, temporality (baseline versus longitudinal, visit schedule), and privacy designations, and may attach transformation functions for forward mapping to the model's feature space and reverse mapping from model outputs to human-readable artifacts.

In some embodiments, synthetic data may be generated to augment minority phenotypes, stress-test pipelines, or support privacy-preserving pretraining without exposing identified records. Inputs may include a schema object, constraint set describing deterministic relations and protocol rules, and seed statistics comprising marginal and pairwise summaries computed from an approved training subset. The transformation may fit a conditional generator that accepts a context vector c encoding visit index, treatment state, and static covariate buckets and that may produce joint samples for the remaining fields. One approach may maintain a pair of networks trained in alternation: a sampler network that may output candidate rows \hat{x} conditioned on c and a critic network that may discriminate between real rows and \hat{x} given c; training may proceed with mini-batches drawn by time slice to preserve temporal co-occurrence structure, and gradient signals may be clipped by feature-wise Lipschitz constraints to limit mode collapse in sparse categories. Following training, a curator may enforce hard constraints by projecting \hat{x} onto the nearest feasible point under a rule engine that may apply unit conversions, monotone repairs, and clinical guardrails). A privacy auditor may compute per-record similarity scores between \hat{x} and the seed subset using locality-sensitive hashing over feature-wise embeddings and may reject samples that exceed a similarity threshold. In some embodiments, a differential privacy tracker may track cumulative privacy budget for published batches. Accepted synthetic cohorts may then pass through the same featurization used for real records so that downstream heads may not depend on data source.

In some embodiments, real-world data may be ingested from electronic health records, claims repositories, registries, or approved data networks and may be prepared to approximate a “target trial” corresponding to the protocol of the ongoing study. Inputs may include raw tables for encounters, procedures, medications, laboratories, outcomes, and death registries, together with a target-trial specification enumerating eligibility rules, index dates, exposure definitions, outcome windows, and censoring rules. The transformation may construct an eligibility mask, compute index times per subject, form time-aligned covariate snapshots, and label treatment and outcomes under the specification. A confounding adjustment module may compute a treatment propensity model and an outcome model on observed data and may construct pseudo-outcomes to support doubly robust estimation when necessary. For longitudinal endpoints, the module may create inverse-probability weights for censoring based on follow-up availability. The resulting summaries may be used to construct external baselines for adverse event rates and to initialize parameters of study-facing heads without forcing the study's primary analyses to depend on observational identification assumptions.

In some embodiments, historical trial data may be incorporated through a hierarchical borrowing layer that may treat historical datasets as exchangeable or partially exchangeable sources relative to the current study. Inputs may include a set of historical trials with their estimands, population descriptors, site distributions, measurement schedules, and coding systems, together with a compatibility function that may score similarity between the current study and each historical dataset along design-level and population-level axes. The transformation may compute a borrowing weight per historical dataset based on the compatibility score and may combine historical summaries with interim summaries to form priors or shrinkage targets for current-study parameters. When the analysis plan may call for prospective dynamic borrowing, a commensurability gate may attenuate the historical contribution if the interim data may appear inconsistent with historical predictions beyond a tolerance.

In some embodiments, the architecture may unify these sources at the featurization boundary by writing a feature store that may compute invariant transforms across sources and attach a source tag that downstream heads may read for domain-shift adjustment. A domain adaptation module may compute per-feature re-weighting or latent alignment to reduce distributional drift between the study cohort and external sources. The platform may thereby preserve the separation between evidence generation and evidence presentation while allowing external evidence to inform initialization, calibration, and predictive simulation in ways that are traceable to specific datasets and alignment transforms.

### Profiling and Anomaly Detection

In some embodiments, a process may be implemented for patient profiling or subgroup profiling, where patients are subgrouped into different categories, e.g. by selector 38. Embodiments may include a module that takes individual patients and, using generative AI (like a large language model (LLM) 24 implemented with a transformer with multi-headed attention), generates a narrative specific to each patient, e.g., based on retrieval augmented generation from data store 20 and outputs of generator 44, along with suitable visualizations (e.g., generated with code and structured data output by the LLM or with a diffusion model). The narrative generation process may involve selecting and inputting various types of data, such as medical history, diagnostic results, and treatment records, into an AI model. Inputs also include metadata about the underlying study datasets and variables that provide further context for the AI model to make even more meaningful use of the data inputted. Further inputs may include human-generated narratives from prior studies and associated data to provide the AI with examples of language and content that those who review narratives are accustomed to seeing in practice.

The AI model for profiling (e.g., in analyzer 46 or model 24) may be a multi-modal model configured to process this data, regardless of its format, whether in the form of graphs, text, tables, or other visual representations. Some embodiments may combine a vision transformer with a transformer configured for natural language processing. The AI profiling model may then analyze the multi-modal data to construct a coherent and comprehensive narrative, which describes the patient's medical journey, identifies key events, and highlights relevant clinical outcomes. In some embodiments, the system may further allow the generated narrative to be summarized into a more concise form, providing a high-level overview of the patient's profile and medical history. The output of the AI-driven narrative generation process may allow DSMB members to quickly understand complex patient data and make informed decisions.

Additionally, some embodiments may incorporate in module 30 machine learning algorithms (or other forms of AI) configured to detect rare events within patient data, such as adverse reactions to treatment. These algorithms may employ anomaly detection techniques, which are expected to identify patterns or occurrences that deviate from the norm. For instance, when an adverse event is detected, the system may assess whether the event is rare by comparing it against a large dataset of similar cases. The system may then attempt to construct a potential explanation for the detected anomaly, although the explanation may or may not be based on causal models. The system may analyze factors such as patient demographics, pre-existing conditions, and treatment regimens to identify correlations that could suggest possible causes for the rare event. Examples of anomaly detection algorithms include the following:
a. The Isolation Forest algorithm may operate by isolating observations in a dataset. It may operate on the principle that anomalies are few and different, and therefore can be isolated. The algorithm may build a forest of random trees by recursively splitting the data points along randomly selected features (e.g. with the classification and regression tree (CART) algorithm), recursively splitting the data to optimize for entropy on each side of the split. Data points that require fewer splits to isolate are more likely to be anomalies. The anomaly score may be computed based on the depth at which an observation is isolated, with lower depths indicating higher likelihoods of being an anomaly.
b. One-Class SVM is a variation of the Support Vector Machine algorithm designed for unsupervised anomaly detection. The algorithm may learn a decision function for a single class, typically representing the “normal” data. It may attempt to separate the normal data points from the origin in the feature space by finding a hyperplane that maximizes the margin from the origin. Data points that fall outside of this boundary (i.e., on the other side of the hyperplane) may be considered anomalies. This approach is particularly useful in scenarios where only normal (non-anomalous) data is available during training.
c. The Local Outlier Factor algorithm may detect anomalies by comparing the density of a point to the densities of its neighbors. For each data point, LOF may calculate the local density, defined as the inverse of the average distance to its k-nearest neighbors. The algorithm then may compute the ratio of the local density of the point to the local densities of its neighbors. A data point may be considered an anomaly if its local density is significantly lower (e.g., more than a threshold amount) than that of its neighbors, indicating that it resides in a region of lower density relative to its surroundings.
d. The k-NN algorithm for anomaly detection may identify anomalies by analyzing the distance of a data point to its nearest neighbors. The expectation, in some embodiments, is that normal data points will have neighbors at similar distances, while anomalies will be far from their nearest neighbors. The anomaly score of a data point may be calculated based on the average distance to its k-nearest neighbors, with larger distances (e.g., more than a threshold) indicating a higher likelihood of being an anomaly. This method is often suitable when the data is well-clustered.
e. The Gaussian Mixture Model is a probabilistic model that assumes the data is generated from a mixture of several Gaussian distributions. The model may estimate the parameters of these Gaussian distributions, such as means and covariances, and may assign a probability to each data point indicating its likelihood of belonging to each distribution. Data points with low likelihoods (e.g., less than a threshold) across all distributions may be flagged as anomalies. GMM is expected to be useful for detecting anomalies in data that is assumed to follow a normal distribution but may include outliers.
f. Autoencoders may be implemented with a type of neural network and may be used for unsupervised anomaly detection, particularly in high-dimensional data. The network may be trained to compress (e.g., encode) the data into a lower-dimensional representation and then reconstruct (e.g., decode) it back to the original dimension. During training, the autoencoder learns to minimize the reconstruction error for normal data, based on an objective function responsive to the difference between the original and reconstructed versions. When an anomaly is inputted, the reconstruction error is expected to be higher because the autoencoder has not learned to reconstruct anomalous patterns well. Anomalies are identified based on the magnitude of this reconstruction error, e.g., designating those greater than a threshold.

Embodiments may further integrate software tools that identify patterns in adverse events, assist with risk stratification, and suggest dose modifications based on patient-specific factors. For instance, the system 12 may analyze data to determine whether a particular dosage is too high for certain subgroups, such as lightweight women, and provide recommendations for dose adjustments. Additionally, the system 12 may be capable of monitoring patient compliance and forecasting the safety and efficacy of ongoing treatments. In some cases, the system 12 may identify patterns where some patients are experiencing adverse effects, and may facilitate discussions around the ethical implications of continuing treatment for specific subgroups. For example, if a drug is associated with adverse effects in a particular demographic, the system may support conversations on whether it is ethical to continue treatment in that subgroup, while considering the risk-benefit tradeoffs.

In some embodiments, the system 12 may be configured to assist DSMB members as they recommend changes to a study's hypothesis, endpoints, or overall design. For instance, based on the data analysis, the DSMB may be empowered to propose changes to the study design, such as selecting different endpoints or targeting a new indication. These recommendations may be part of the DSMB's charter, which may outline their authority to make such changes, with the sponsor remaining blinded to certain data while the DSMB has full visibility. To this end, the system 12 may also support the production of networks that identify subpopulations, generate patient narratives, and forecast safety and compliance outcomes, thereby guiding DSMB members in fulfilling their roles effectively.

In some embodiments, tools like OpenAI's Code Interpreter™ may be integrated into the platform to further enhance its capabilities in analyzing and interpreting complex datasets within the context of clinical trials and patient profiling. The platform may write, execute, and modify custom code with generative AI models, facilitating more advanced and tailored data analysis. In some cases, AI agents may be configured in a pipeline with a shared goal and different sub-tasks pursuant to that goal.

For instance, when examining patterns in adverse events, the system 12 may be used to generate code for and run algorithms that are not hard-coded into the platform, e.g., by engaging model 24 and executing in environment 32 based on data from data store 20. Users may request generation of scripts to perform complex statistical analyses, such as advanced anomaly detection or rare event modeling, which may involve statistical methods or machine learning models.

The system 12 may also support the customization of visualizations and annotations, e.g, with generator 44. For example, AI models, like LLMs (like model 24) trained for code generation, may write scripts executed in environment 32 to generate unique visual representations of data, such as heatmaps, scatter plots, or interactive dashboards, that better capture the nuances of the patient data or clinical trial results. These custom visualizations can be annotated within the platform in some embodiments, facilitating precise and data-driven discussions.

Embodiments may also allow for the implementation of custom models for risk stratification and dose modification analysis. Users may, with the aid of AI-generated code for example, develop and test predictive models that incorporate various patient-specific factors, such as demographic information, comorbidities, and genetic data, to assess the risk of adverse events and recommend personalized dose adjustments.

### Heterogenous Treatment Effects

In some cases, brute force approaches taken by some computing systems may not be suitable for finding heterogenous treatment effects in candidate subpopulations of patients. As more patient characteristics are added, the space of possible subgroups grows combinatorially. For instance, with only 20 such features of each patient, there are over a million permutations of subgroups. If a study allows for continuous variables like age or weight, each possible cut-point multiplies the number of candidate subgroups even further. A brute force strategy that tries every combination of covariates and thresholds quickly becomes computationally infeasible, both because of the sheer number of possible splits and because each subgroup needs enough patients for reliable effect estimation. Brute force enumeration may also be statistically fragile, as most subgroups could contain very few patients, making it impossible to estimate treatment effects reliably.

To mitigate these issues, some embodiments have algorithms for heterogeneous treatment effect discovery focus on adaptively searching for informative subgroups rather than exhaustively enumerating them. In some embodiments, individual treatment effect estimates may be obtained by modeling the conditional average treatment effect as a function of patient covariates. These covariates may include continuous variables (e.g., age, weight, laboratory values), categorical variables (e.g., gender, race, comorbidity categories), or high-cardinality categorical or ordinal variables (e.g., diagnostic codes, medication classes). Estimation of conditional average treatment effect τ(x) may be performed using one or more meta-learning approaches, where base models are trained on subproblems derived from the treatment and control arms of the trial. In some embodiments, a T-learner approach may involve training two independent regression models, one for each treatment arm, and computing τ(x) as the difference in predicted outcomes for a given x. In contrast, in some embodiments an S-learner may be trained on the full dataset using a single model that includes treatment as an input feature, and τ(x) may be estimated by computing counterfactual predictions for both treatment assignments. In some embodiments, an X-learner may first impute counterfactuals using the T-learner outputs and then use these to train a model that predicts τ(x) directly. In some embodiments, doubly robust learners such as DR-learner or R-learner may be employed, where outcome and propensity models are both estimated and combined to compute residualized outcomes or pseudo-outcomes. For example, in a DR-learner implementation, cross-fitting may be used to obtain out-of-fold predictions of potential outcomes and propensity scores, and residuals from these models may be regressed on covariates x to estimate τ(x) with reduced bias under misspecification of either the outcome or the treatment model. Base models in these meta-learners may include gradient boosted machines, random forests, elastic net penalized regressions, or Bayesian Additive Regression Trees (BART), among others. In some embodiments, causal forest models such as generalized random forests may be used to estimate τ(x) directly via an ensemble of trees that use an honest splitting procedure, where data used to determine split points is separate from data used to estimate within-node effects. Such forests may handle mixed-type covariates and may provide estimates of treatment effect variance by aggregating across trees trained on disjoint subsets of data.

In some embodiments involving time-to-event outcomes, treatment effect heterogeneity may be modeled by extending causal forests to survival settings (causal survival forests), where splitting criteria may target differences in cumulative hazard functions or restricted mean survival time between treatment groups, stratified by covariates. Alternatively, parametric or semiparametric survival models such as Cox proportional hazards models or accelerated failure time models may be augmented with treatment-by-covariate interaction terms, optionally subject to sparsity-inducing regularization. For instance, in some embodiments, interaction terms between treatment indicators and individual covariates or higher-order interaction terms may be included in a penalized likelihood framework, such as clastic net or hierarchical lasso, which may enforce inclusion of main effects when corresponding interactions are retained.

After obtaining \hat{τ}(x) and its associated standard error or posterior uncertainty estimates, some embodiments may convert the treatment effect estimates into actionable subgroup labels by applying decision rules to the \hat{τ}(x) distribution. In some embodiments, a thresholding rule of the form \hat{τ}(x)−k·SE(\hat{τ}(x))>0 may be used to identify patient profiles predicted to benefit from treatment with high confidence, where k is a user-specified quantile multiplier reflecting a desired false discovery rate. Conversely, profiles for which \hat{τ}(x)+k·SE(\hat{τ}(x))≤0 may be labeled as non-beneficiaries, and remaining cases may be labeled as indeterminate. In some embodiments, policy learning techniques may be applied to learn a treatment assignment policy π(x) that maps covariates to a binary treatment decision based on predicted benefit. For binary outcomes, evaluation of π(x) may be conducted using metrics such as the area under the uplift curve (AUUC) or the Qini coefficient. In some embodiments, policy value may be estimated using inverse-propensity weighting or doubly robust estimators applied to held-out data folds.

In some embodiments, interpretable subgroups may be extracted from \hat{τ}(x) by compressing model predictions into rule-based representations. These may include tree-based models such as uplift trees or causal trees, which split the covariate space based on treatment effect heterogeneity criteria. Alternatively, rule list models such as RuleFit, Bayesian Rule Lists, or Falling Rule Lists may be trained on \hat{τ}(x) predictions to derive short conjunctions of covariate conditions (e.g., “Age >70 and Chronic Kidney Disease and BMI <22”) that approximate the \hat{τ}(x) mapping. In some embodiments, methods such as QUINT (Qualitative Interaction Trees) may be used to identify covariate combinations associated with qualitative treatment effect reversals. Classical subgroup discovery frameworks such as SIDES (Subgroup Identification based on Differential Effect Search), Virtual Twins, or GUIDE (Generalized, Unbiased, Interaction Detection and Estimation) may also be applied in certain embodiments.

To address multiplicity, control over false discovery rate (FDR), family-wise error rate (FWER), or selective inference bias may be incorporated into the subgroup discovery process, e.g., by selector 38. In some embodiments, candidate subgroups identified on a discovery fold may be evaluated on a separate confirmation fold, and subgroup-specific p-values may be adjusted using permutation tests or stepwise multiplicity control procedures. When missingness or noncompliance may bias heterogeneity estimates, some embodiments may report bounds or conduct sensitivity analysis under various assumptions. In some embodiments, model-consistent multiple imputation may be applied, such as imputing missing covariates using models trained on observed data that match the structural assumptions of the \hat{τ}(x) model. To assess the stability of selected subgroups or decision rules, bootstrap sampling or subsampling may be used to estimate rule selection frequencies or variation in effect estimates across samples.

In some embodiments involving survival outcomes, treatment effect heterogeneity may be summarized in terms of contrasts in restricted mean survival time (RMST) across subgroups defined by \hat{τ}(x), allowing time-based interpretations of benefit. For longitudinal outcomes with repeated measurements, mixed-effects models may include treatment-by-time-by-covariate interactions, and effect heterogeneity may be modeled dynamically across timepoints. In cases involving recurrent events, counting process representations may be extended to include covariate-modulated treatment effects, either in a parametric or semiparametric framework.

Throughout the workflow, out-of-sample validation may be applied to some or all stages. In some embodiments, \hat{τ}(x) estimates may be stratified into quantiles or deciles, and observed treatment differences within each stratum may be plotted against average \hat{τ}(x) values to assess calibration. For clinical interpretation, net benefit or decision curve analysis may be conducted to compare treatment assignment policies based on subgroup rules versus default strategies. In some embodiments, final output may include both rule-based subgroup assignments and model-based treatment effect scores, each accompanied by confidence intervals or posterior credible intervals quantifying uncertainty.

### Determining Candidate Causes

In some embodiments, a rare-event analysis module of generator 36 may receive as input a stream of anchored records describing patient demographics, pre-existing conditions, treatment regimens, longitudinal measurements, and adjudicated event logs, and may produce as output a ranked set of factor-event associations with uncertainty descriptors and provenance pointers. Demographics may include age at index date, sex, race, ethnicity, and site or region codes. Pre-existing conditions may be derived from diagnostic coding systems and problem lists with look-back windows. Treatment regimens may be represented as sequences of administrations, dose amounts, dose intensity summaries, interruptions, and concomitant medications. Longitudinal measurements may include laboratory and vital sign trajectories aligned to visit schedules. Event logs may include timestamps, grades, seriousness flags, and outcomes for the rare event class under study. Each feature and outcome instance may carry an anchor that may allow reverse navigation to the underlying dataset row and the corresponding visible element in reports.

In some embodiments, the module in generator 36 may construct time-at-risk windows relative to candidate triggers such as first exposure, dose escalation, or concomitant initiation. A labeling stage may assign, for each patient and window, an outcome flag describing whether the rare event occurred within the window and a censoring flag describing follow-up completeness. A featurization stage may compute baseline summaries (for example, pre-index averages and slopes), contemporaneous summaries within the window (for example, rolling means and threshold breaches), and regimen descriptors (for example, cumulative dose to date, relative dose intensity, and recent changes), each stored with units and missingness indicators. Where a factor can vary over time, the featurization stage may materialize multiple snapshots aligned with discrete visit or day indices so that subsequent analyses may consider temporal precedence.

In some embodiments, a correlation discovery stage implemented by generator 36 may proceed along three coordinated tracks to address sparsity. A pooled association track may compute disproportionality statistics between candidate factors and the rare event using contingency counts derived from the labeled window. This track may include exact or permutation-based significance computations when cell counts may be small. A temporal association track may evaluate whether onsets may cluster at characteristic lags following changes in a factor by scanning factor change points against event times and computing enrichment relative to a null obtained by time reshuffling that may preserve within-patient visit spacing. A self-controlled track may restrict analysis to patients who may have both exposed and unexposed intervals and may compare event incidence across those intervals, which may reduce confounding by time-invariant patient characteristics. The three tracks may produce association scores that the system may later combine in a meta-analytic fashion while preserving anchors for all contributing counts and intervals.

In some embodiments, a regression layer may test whether candidate factors may retain association with the rare event after accounting for measured covariates. The layer may construct a design matrix that may include demographics, comorbidity indicators, regimen descriptors, and recent longitudinal summaries, and may fit a sparse classifier or survival model appropriate for the windowing scheme. For windowed binary outcomes, the layer may train a penalized logistic model that may select a subset of factors while mitigating overfitting. For time-to-event analyses, the layer may train a discrete-time hazard model with interval indicators so that time granularity may match the visit schedule. Regularization strengths and feature groupings may be selected through cross-validation stratified by site or cohort. The layer may output adjusted association measures with uncertainty intervals and may write model diagnostics including calibration curves, residual checks, and stability summaries across resamples.

In some embodiments, a pattern mining model of generator 36 may search for interactions among factors that may precede the rare event. The model may generate candidate conjunctions by mining frequent closed itemsets within event-positive windows under minimum support tuned for rarity and may filter candidates using temporal precedence rules that may require that each constituent factor change precede the event by at least a configured lag. Surviving candidates may be evaluated in held-out folds using small generalized models with interaction terms to estimate whether the conjunction may improve fit beyond main effects. The model may emit human-readable rules tied to the contributing records and with estimates of incremental association and uncertainty.

In some embodiments, a causal plausibility screen implemented by generator 36 may reduce spurious correlations before hand-off to decision makers. The screen may compute a propensity for exposure to each modifiable factor using baseline covariates and may derive residualized outcomes or pseudo-outcomes that may reduce bias when treatment assignment may not be purely randomized. The screen may re-estimate factor-event associations on these residualized targets and may flag candidates whose apparent association may be sensitive to the adjustment choice, cohort definition, or time-at-risk construction. A sensitivity bundle may accompany each flagged association and may include results under alternative windows, exclusion of specific sites, and down-weighting of extreme propensity scores, together with a stability field describing the frequency with which the association may be rediscovered across bootstrap resamples.

In some embodiments, the generator 36 may incorporate mechanistic or protocol-defined guardrails when associating treatment regimens with rare events. Dose change events, holds, or re-challenges may be encoded as discrete actions, and the analysis may distinguish correlations with absolute dose levels from correlations with dose transitions. Concomitant medications may be represented as overlapping intervals, and an overlap engine may compute pairwise and triple overlaps with treatment windows and event windows so that correlations may consider co-exposures explicitly. Where laboratory thresholds may define suspected mechanisms, the module may include threshold-crossing indicators and recovery times as candidate factors and may analyze their temporal alignment with the rare event.

In some embodiments, the ranking and reporting stage of generator 36 may combine evidence from the pooled, temporal, self-controlled, regression, and pattern mining tracks. A rank aggregator may compute a composite score per factor or factor set by normalizing track-specific statistics to comparable scales and averaging with weights that may reflect internal data quality metrics such as follow-up completeness and site heterogeneity. The stage may apply multiplicity control across the evaluated hypotheses and may emit, for each factor, a record that may include the composite score, per-track statistics, adjusted uncertainty descriptors, sensitivity summaries, and the list of anchors that may reproduce the underlying counts and time alignments. The record may also store a suggested follow-up analysis plan, such as replication in a discovery-confirmation split or targeted chart review, without asserting any preference among options.

In some embodiments, the generator 36 may surface the resulting association artifacts in patient- and cohort-level views as linked evidence cards. A DSMB reviewer may select a factor to see per-patient timelines highlighting when the factor may have changed relative to event onset, the window-specific risk deltas predicted by the adjusted models, and the original anchor tokens embedded in the source tables or reports. A reconciliation routine may monitor whether subsequent data refreshes may alter the counts or time alignments that supported a correlation and may annotate the artifact with a change log when estimates may shift, allowing discussions and minutes to reference the exact computational context in which a correlation was observed.

### Multichannel UI/UX

Some embodiments require no coding knowledge, allowing users to interact with the system through a point-and-click interface or voice interface, e.g., via a speech-to-text model. The front end of the platform may be designed to be intuitive, enabling DSMB members to easily navigate and manipulate data, conduct analyses, and make informed decisions.

The system 12 may allow users to manage large datasets by focusing on specific fields of interest. For example, a DSMB user might request the generation of detailed patient profiles for every individual within a study, facilitating a thorough review of each patient's data. This functionality may be integrated into the system's 12 workflow, allowing DSMB users to produce and review patient profiles, identify significant patterns, and discuss the implications in real-time. If a study shows adverse effects related to a drug, the platform may support discussions on the ethics of continuing the study, particularly for specific subgroups where the risk may outweigh the potential benefits.

The system 12 may provide access to a comprehensive dataset, allowing users to explore within-study data in relation to external datasets. For example, the system 12 may help determine whether observed adverse events are truly indicative of a signal or merely background noise. Outputs from DSMB meetings, such as voting on whether to continue or modify a study, may also be documented by the platform. These outputs may include recommendations to discontinue specific doses, submit data based on observed efficacy, or address safety concerns. The platform may also support the generation of meeting minutes from quarterly DSMB meetings (e.g., by inputting an audio feed of the same into a large language model and requesting a summary), tracking action items and workflows to ensure accountability and follow-up on critical decisions.

In some embodiments, the system 12 (e.g., through manager 42) may be configured to facilitate data-driven conversations through the use of visualizations and annotation tools. This system 12 may help DSMB users to engage in discussions directly within the context of the data being analyzed. For example, the system 12 may display patient profiles, treatment outcomes, and other relevant data through various visual formats such as graphs, charts, and annotated timelines. Users, such as clinical trial stakeholders or DSMBs, may interact with the data by adding annotations, highlighting specific trends or concerns, and sharing their insights in real-time. This contextual interaction is expected to improve the quality and relevance of discussions, allowing for more informed decision-making processes.

Some embodiments provide a unified user interface and user experience that allows a DSMB to work seamlessly across structured data formats (such as CSV), rendered documents (such as PDF), handwritten annotations from paper or tablets, and programmatic comments originating from environments such as SAS or Jupiter. The system 12, in some embodiments, synthesizes these inputs into a cohesive, citable DSMB report while maintaining consistent support for AI-assisted safety review, patient profiling, visualization of outcomes, and contextual discussions.

In some embodiments, clinical datasets from data store 20 and all derived representations (like tables, charts, PDFs, patient narratives) may be modeled as nodes in a shared artifact graph of a trial. The primary node of that graph may correspond to a versioned dataset, such as Analysis Data Model (ADaM) or Study Data Tabulation Model (SD™) tables imported from CSV. From this dataset, the platform may generate derived “view” nodes including patient profile summaries, population-level tables, interactive charts, and printable PDF packets for meetings. Each element within a generated view may be associated with a stable data anchor that encodes its lineage back to the raw data. For example, a table cell may be anchored using a tuple such as {dataset_id, subject_id, visit_number, variable_name, timestamp}, and a charted data point may be similarly anchored to its originating row and field. These anchors may be embedded invisibly into PDFs using metadata formats such as Extensible Metadata Platform (XMP), and also printed alongside visual artifacts using QR micro-tags or compact hash codes, allowing downstream optical capture and re-anchoring of screenshots, scan-based annotations, or stylus inputs. This structure, in some embodiments, is expected to support navigation and traceability across AI-generated summaries, safety signals, and linked source data.

In some embodiments, a “Data Mode” interface may present a spreadsheet-like grid for users working with structured data formats. This grid may include schema-aware validation, column metadata, and saved filter conditions. Selecting a cell, row, or cohort in this mode may open synchronized panels, including a patient narrative view, associated visualizations, and an anchored conversation drawer scoped to the selected data anchor. In parallel, a “Report Mode” may display paginated PDF content with a section navigation sidebar and evidence highlights. Selecting any element in the PDF (e.g., a value, figure, or label) may resolve its embedded anchor and invoke the corresponding conversation drawer. This design, in some embodiments, may allow users with different workflows, including PDF-centric and data-centric reviewers, to collaborate using a shared anchor model while preserving a unified discussion space per data element.

Some embodiments support ingestion of paper-based annotations by applying page fingerprinting, optical character recognition (OCR), handwriting recognition, and shape detection. Printed micro-tags and embedded identifiers in the PDF may be used to re-anchor annotations such as drawn circles, margin notes, or arrows. When ambiguous mappings occur (e.g., multiple candidate anchors exist for a single hand-marked area) the interface may prompt the reviewer to disambiguate and then cache that resolution for future consistency. Scanned pages may be shown with overlays of detected ink and annotation context in a threaded comment panel filtered to inferred anchors. This interaction allows continued use of “print-mark-scan” workflows while maintaining linkage to live, versioned data.

For tablet-based review sessions, stylus input may be captured as vector ink with metadata such as stroke color, pressure, and page coordinates. Each stroke may be immediately associated with the closest visual anchor at the time of capture. This allows reviewers to annotate graphical plots, call out individual lab values, or draw attention to sections of Kaplan-Meier survival curves. Users may toggle between a clean PDF and an annotated overlay, and interface controls may allow updating the thread status (e.g., open, requires analysis, resolved, include in minutes). Lightweight voting mechanisms may be provided, and these interactions may feed into the structured decision logging contemplated for DSMB sessions.

For programmatic users, lightweight integration points may be provided. In SAS, this may take the form of a macro or tagset that outputs a JSON sidecar or posts to an API with parameters such as dataset identifier, subject ID, variable name, and a comment string. In Jupyter, a cell magic directive may allow analysts to attach notes to specific rows of displayed DataFrames, plotted points in a Matplotlib figure, or embedded patient cards. The platform may inject invisible data attributes or metadata alongside visual elements to preserve these anchors. When such outputs are rendered inside the application's notebook previewer, clicking an element may automatically invoke the comment drawer at the correct anchor.

Annotations from any of these sources may be consolidated into “evidence cards.” Each evidence card may represent a specific anchor and aggregate all commentary, markups, highlights, and model-derived insights linked to that anchor. Cards may display the live value of interest along with metadata such as confidence intervals, model uncertainty scores, historical values across data versions, narrative excerpts, and automated anomaly detection outputs. Navigation from an evidence card may allow deep-linking into the data grid, PDF report, patient profile view, or corresponding visualization. This structure supports contextual analysis of adverse events, risk patterns, or unexpected treatment outcomes.

During live DSMB meetings, some embodiments may offer a structured “Session” view that sequences the PDF packet, allocates time for each section, and records decisions directly attached to relevant anchors. An audio feed may be transcribed to support automated drafting of meeting minutes. These drafts may include traceable references back to anchors and evidence cards cited during discussion. A live checklist interface may track quorum, declared conflicts, motions, and votes, and a post-meeting module may assemble a signed PDF of the meeting minutes with provenance trails for each entry.

To support traceability and data reconciliation, the system 12 may flag discrepancies when an annotated value no longer matches the current dataset. For example, if a reviewer circles an alanine transaminase (ALT) value of 182 on a printed sheet but the live dataset reflects 178, the UI may show a discrepancy banner on the corresponding evidence card. Additional actions may include accepting the reviewer's annotation, flagging a suspected transcription error, or initiating a data quality workflow. Each action may be logged with timestamps, user identifiers, and rationale for audit compliance and selective inference tracking.

In some embodiments, vision models may identify table structures and associate ink annotations with data anchors. Language models (like model 24) may summarize threaded conversations, generate candidate minutes, and draft patient narratives. Statistical or policy-learning models may estimate subgroup-level treatment effects and visualize these as ribbon overlays on evidence cards. In some cases, model outputs may be paired with uncertainty indicators and human-sourced notes, with clear visual separation between system-generated and reviewer-authored content.

Export functionality may maintain data traceability. CSV exports may be accompanied by sidecar JSON files that include thread references and decisions for each row. PDF exports may preserve embedded anchors and optionally render annotations into the visible layer, allowing round-trip re-ingestion while maintaining auditability. The DSMB report generator 44 may compile sections backed by evidence cards and their linked anchors, assembling a structured narrative that includes both machine-generated drafts and human-approved edits. This report may allow navigation from each recommendation or conclusion back to the exact underlying values, figures, and discussion threads that supported it.

In some embodiments, patient profiles may be emitted as structured, per-patient objects that fuse static covariates, longitudinal measurements, risk and forecast heads, and a decision log. The object may include sections for demographics, eligibility flags, disease history, dosing timeline with exposure summaries, laboratory and vital trends with thresholds, adverse event history with grades and outcomes, and links to imaging or specialty assessments. Each section entry may carry a direct anchor to the source table row and a lineage record documenting all transformations applied. A profile assemble in generator 44 may receive a list of anchors for a patient and a visit window and may compute a time-aligned panel. Generator 44 may then attach model outputs relevant to the window, including current risk vectors, forecast bands, and any dose policy recommendations with their constraint audits.

In some embodiments, patient narrative outputs may include natural-language summaries derived from the profile object and associated discussions. A content selection stage may extract salient events and trends by ranking candidates with learned importance scores derived from model attributions, deviation magnitudes, and discussion activity around matching anchors. A sentence planning stage may order selected items by clinical storyline, optionally grouping by organ system or visit. A surface realization stage may render sentences from templates with slot filling, with language-model refinement constrained by anchor-preserving placeholders and rule-based guards for numerics and units. In some cases, each mention of a value or event may embed an invisible anchor token in the rendering payload.

In some embodiments, some risk assessment outputs may be emitted as calibrated probability vectors and decision-rule evaluations at one or more time horizons. Each vector element (or some) may correspond to a defined event type or boundary rule (for example, Grade ≥2 liver toxicity within 30 days), accompanied by calibration diagnostics such as reliability bin statistics and Brier components, as well as fairness diagnostics computed over prespecified subgroup labels. Threshold applications may be stored explicitly as rule evaluations with fields {rule_id, threshold, estimate, uncertainty, pass/fail, anchors}. Where discrete-time survival heads may be used, the output may additionally include stepwise hazards and integrated cumulative incidence for the configured horizon.

In some embodiments, forecast outputs may provide multi-horizon predictions for clinical measurements, adverse event hazards, endpoint status, or restricted mean survival time. Time-series forecasts may be emitted as arrays of means and quantiles per horizon step with correlation descriptors that may allow simulation of joint future paths; event forecasts may include hazard or odds trajectories and corresponding cumulative curves; endpoint forecasts may include probability-of-response at scheduled assessment visits. Scenario variants may be included by conditioning the rollout on candidate treatment actions or adherence states. These forecasts may be linked into decision modules and meeting packets through the same anchor system as other outputs so that each plotted point or table cell may resolve to its provenance and uncertainty context.

In some embodiments, a secure portal may be provided by system 12 to workstations 18, where the portal may surface trial information, allow anchored commenting, and record votes as structured artifacts. Client devices, like workstations 18, may include desktop and laptop computers, tablets, and mobile telephones running native or browser-based user interfaces that query a server over one or more networks; the server may host a database storing trial data and rendered artifacts consumable by the portal. The portal may present patient profiles, treatment outcomes, graphs, charts, and annotated timelines, and may allow interaction with these views through in-context annotations and threaded discussion. In some embodiments, outputs from DSMB meetings, including votes to continue or modify a study, may be captured by the portal as records linked to the discussed evidence.

In some embodiments, user authentication may be performed via an authorization server that may issue short-lived bearer tokens after multi-factor verification. The portal may maintain a per-session cryptographic context and may attach a role- and attribute-based authorization envelope to each request so that the server may evaluate access policies down to the column or anchor level. A request dispatcher may verify token validity, evaluate policy rules, and route to a read path for artifact retrieval or a write path for comments, annotations, or votes.

In some embodiments, the portal may render a synchronized artifact graph that may include versioned dataset nodes and view nodes such as tables, charts, patient profiles, and printable packets, where each visible element may carry a stable anchor that links to record-level data and corresponding evidence cards. When a DSMB member selects a value or figure in the portal, the interface may resolve its anchor and open an anchored conversation drawer scoped to that element so that comments may reference precisely the underlying source. The same anchored conversation may be accessible whether the user is in a spreadsheet-like data grid, a patient-profile view, or a paginated report view, maintaining one source of truth about the discussion context.

In some embodiments, commenting may be implemented as a real-time, anchor-scoped thread model. Each thread object may include fields {thread_id, anchor, participants[ ], visibility, status, timestamps, message_list[ ]}, and each message may include {message_id, author_id, body_richtext, quoted_anchor?, attachments[ ], redlines?}. The client may maintain a local operation log for comment edits and reactions and may synchronize with the server using a conflict-free replicated data type so that concurrent edits by multiple DSMB members may converge without loss; server-side ordering may be recorded using hybrid logical clocks so that a deterministic timeline may be reconstructed. The portal may support reply, resolve, reopen, and “add to minutes” actions as state transitions on the thread object, and each transition may be written to the audit hash chain.

In some embodiments, voting may be modeled as motions with typed ballots and quorum logic. A motion object may include {motion_id, title, description, anchors[ ], ballot_type, eligible_voters[ ], quorum_rule, pass_rule, blind?, start_time, end_time, status}, and a ballot may include {voter_id, selectionE {approve, reject, abstain, hold, alternative options}, justification_text, conflict_of_interest_declaration, signature}. The portal may present active motions in a session view and may prevent ballot submission by users who have not acknowledged conflicts or who are not marked eligible by the charter configuration. Upon submission, the client may compute a detached digital signature over the ballot payload and transmit both payload and signature; the server may verify the signature, persist the ballot, and recompute the motion state under the charter's quorum and pass rules. Voting outcomes and recommendations may be documented by the portal for later inclusion in minutes.

In some embodiments, the portal may support meeting workflows by sequencing pre-read packets, time-boxing sections, and attaching decisions to anchors in real time. A live session controller may orchestrate agenda progression, enforce time windows, and expose a checklist of quorum attainment, conflicts, motions, and votes. When an audio feed may be ingested for draft minutes, the portal may tie transcript segments to anchors and threads that participants referenced, allowing later review to reproduce the context in which each comment or vote occurred.

In some embodiments, transport and storage security may include channel encryption, token-bound session cookies, and encryption-at-rest with data keys rotated per project. The portal may watermark rendered documents with user-specific tokens, may restrict copy/print operations for sensitive views, and may display provenance banners that indicate dataset version and analysis model used to generate each artifact. For offline use, the portal may maintain an encrypted cache for read-only artifacts and queued write operations; upon reconnection, a synchronization routine may replay queued operations against server policies and reconcile conflicts via the same replicated data structure used for real-time edits.

### Example Computing Device

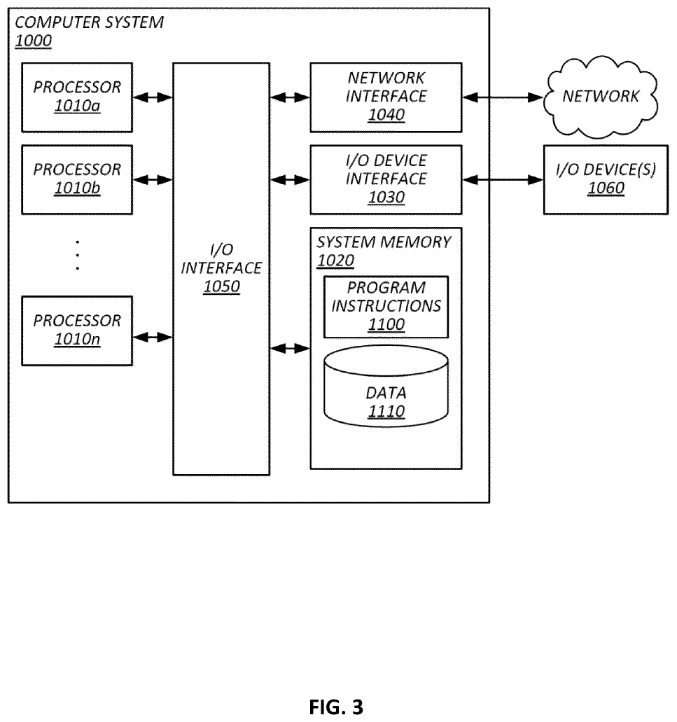

FIG. 3 is a diagram that illustrates an exemplary computing system 1000 in accordance with embodiments of the present technique. A single computing device is shown, but some embodiments of a computer system may include multiple computing devices that communicate over a network, for instance in the course of collectively executing various parts of a distributed application. Various portions of systems and methods described herein, may include or be executed on one or more computer systems similar to computing system 1000. Further, processes and modules described herein may be executed by one or more processing systems similar to that of computing system 1000.

Computing system 1000 may include one or more processors (e.g., processors 1010a-1010n) coupled to system memory 1020, an input/output I/O device interface 1030, and a network interface 1040 via an input/output (I/O) interface 1050. A processor may include a single processor or a plurality of processors (e.g., distributed processors). A processor may be any suitable processor capable of executing or otherwise performing instructions. A processor may include a central processing unit (CPU) that carries out program instructions to perform the arithmetical, logical, and input/output operations of computing system 1000. A processor may execute code (e.g., processor firmware, a protocol stack, a database management system, an operating system, or a combination thereof) that creates an execution environment for program instructions. A processor may include a programmable processor. A processor may include general or special purpose microprocessors. A processor may receive instructions and data from a memory (e.g., system memory 1020). Computing system 1000 may be a uni-processor system including one processor (e.g., processor 1010a), or a multi-processor system including any number of suitable processors (e.g., 1010a-1010n). Multiple processors may be employed to provide for parallel or sequential execution of one or more portions of the techniques described herein. Processes, such as logic flows, described herein may be performed by one or more programmable processors executing one or more computer programs to perform functions by operating on input data and generating corresponding output. Processes described herein may be performed by, and apparatus can also be implemented as, special purpose logic circuitry, e.g., an FPGA (field programmable gate array) or an ASIC (application specific integrated circuit). Computing system 1000 may include a plurality of computing devices (e.g., distributed computer systems) to implement various processing functions.

I/O device interface 1030 may provide an interface for connection of one or more I/O devices 1060 to computer system 1000. I/O devices may include devices that receive input (e.g., from a user) or output information (e.g., to a user). I/O devices 1060 may include, for example, graphical user interface presented on displays (e.g., a cathode ray tube (CRT) or liquid crystal display (LCD) monitor), pointing devices (e.g., a computer mouse or trackball), keyboards, keypads, touchpads, scanning devices, voice recognition devices, gesture recognition devices, printers, audio speakers, microphones, cameras, or the like. I/O devices 1060 may be connected to computer system 1000 through a wired or wireless connection. I/O devices 1060 may be connected to computer system 1000 from a remote location. I/O devices 1060 located on remote computer system, for example, may be connected to computer system 1000 via a network and network interface 1040.

Network interface 1040 may include a network adapter that provides for connection of computer system 1000 to a network. Network interface 1040 may facilitate data exchange between computer system 1000 and other devices connected to the network. Network interface 1040 may support wired or wireless communication. The network may include an electronic communication network, such as the Internet, a local area network (LAN), a wide area network (WAN), a cellular communications network, or the like.

System memory 1020 may be configured to store program instructions 1100 or data 1110. Program instructions 1100 may be executable by a processor (e.g., one or more of processors 1010a-1010n) to implement one or more embodiments of the present techniques. Instructions 1100 may include modules of computer program instructions for implementing one or more techniques described herein with regard to various processing modules. Program instructions may include a computer program (which in certain forms is known as a program, software, software application, script, or code). A computer program may be written in a programming language, including compiled or interpreted languages, or declarative or procedural languages. A computer program may include a unit suitable for use in a computing environment, including as a stand-alone program, a module, a component, or a subroutine. A computer program may or may not correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one or more computer processors located locally at one site or distributed across multiple remote sites and interconnected by a communication network.

System memory 1020 may include a tangible program carrier having program instructions stored thereon. A tangible program carrier may include a non-transitory computer readable storage medium. A non-transitory computer readable storage medium may include a machine readable storage device, a machine readable storage substrate, a memory device, or any combination thereof. Non-transitory computer readable storage medium may include non-volatile memory (e.g., flash memory, ROM, PROM, EPROM, EEPROM memory), volatile memory (e.g., random access memory (RAM), static random access memory (SRAM), synchronous dynamic RAM (SDRAM)), bulk storage memory (e.g., CD-ROM and/or DVD-ROM, hard-drives), or the like. System memory 1020 may include a non-transitory computer readable storage medium that may have program instructions stored thereon that are executable by a computer processor (e.g., one or more of processors 1010a-1010n) to cause the subject matter and the functional operations described herein. A memory (e.g., system memory 1020) may include a single memory device and/or a plurality of memory devices (e.g., distributed memory devices). Instructions or other program code to provide the functionality described herein may be stored on a tangible, non-transitory computer readable media. In some cases, the entire set of instructions may be stored concurrently on the media, or in some cases, different parts of the instructions may be stored on the same media at different times.

I/O interface 1050 may be configured to coordinate I/O traffic between processors 1010a-1010n, system memory 1020, network interface 1040, I/O devices 1060, and/or other peripheral devices. I/O interface 1050 may perform protocol, timing, or other data transformations to convert data signals from one component (e.g., system memory 1020) into a format suitable for use by another component (e.g., processors 1010a-1010n). I/O interface 1050 may include support for devices attached through various types of peripheral buses, such as a variant of the Peripheral Component Interconnect (PCI) bus standard or the Universal Serial Bus (USB) standard.

Embodiments of the techniques described herein may be implemented using a single instance of computer system 1000 or multiple computer systems 1000 configured to host different portions or instances of embodiments. Multiple computer systems 1000 may provide for parallel or sequential processing/execution of one or more portions of the techniques described herein.

Those skilled in the art will appreciate that computer system 1000 is merely illustrative and is not intended to limit the scope of the techniques described herein. Computer system 1000 may include any combination of devices or software that may perform or otherwise provide for the performance of the techniques described herein. For example, computer system 1000 may include or be a combination of a cloud-computing system, a data center, a server rack, a server, a virtual server, a desktop computer, a laptop computer, a tablet computer, a server device, a client device, a mobile telephone, a personal digital assistant (PDA), a mobile audio or video player, a game console, a vehicle-mounted computer, or a Global Positioning System (GPS), or the like. Computer system 1000 may also be connected to other devices that are not illustrated, or may operate as a stand-alone system. In addition, the functionality provided by the illustrated components may in some embodiments be combined in fewer components or distributed in additional components. Similarly, in some embodiments, the functionality of some of the illustrated components may not be provided or other additional functionality may be available.

Those skilled in the art will also appreciate that while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (e.g., as instructions or structured data) on a computer-accessible medium or a portable article to be read by an appropriate drive, various examples of which are described above. In some embodiments, instructions stored on a computer-accessible medium separate from computer system 1000 may be transmitted to computer system 1000 via transmission media or signals such as electrical, electromagnetic, or digital signals, conveyed via a communication medium such as a network or a wireless link. Various embodiments may further include receiving, sending, or storing instructions or data implemented in accordance with the foregoing description upon a computer-accessible medium. Accordingly, the present techniques may be practiced with other computer system configurations.

In block diagrams, illustrated components are depicted as discrete functional blocks, but embodiments are not limited to systems in which the functionality described herein is organized as illustrated. The functionality provided by each of the components may be provided by software or hardware modules that are differently organized than is presently depicted, for example such software or hardware may be intermingled, conjoined, replicated, broken up, distributed (e.g. within a data center or geographically), or otherwise differently organized. The functionality described herein may be provided by one or more processors of one or more computers executing code stored on a tangible, non-transitory, machine readable medium. In some cases, notwithstanding use of the singular term “medium,” the instructions may be distributed on different storage devices associated with different computing devices, for instance, with each computing device having a different subset of the instructions, an implementation consistent with usage of the singular term “medium” herein. In some cases, third party content delivery networks may host some or all of the information conveyed over networks, in which case, to the extent information (e.g., content) is said to be supplied or otherwise provided, the information may be provided by sending instructions to retrieve that information from a content delivery network.

The reader should appreciate that the present application describes several independently useful techniques. Rather than separating those techniques into multiple isolated patent applications, applicants have grouped these techniques into a single document because their related subject matter lends itself to economies in the application process. But the distinct advantages and aspects of such techniques should not be conflated. In some cases, embodiments address all of the deficiencies noted herein, but it should be understood that the techniques are independently useful, and some embodiments address only a subset of such problems or offer other, unmentioned benefits that will be apparent to those of skill in the art reviewing the present disclosure. Due to cost constraints, some techniques disclosed herein may not be presently claimed and may be claimed in later filings, such as continuation applications or by amending the present claims. Similarly, due to space constraints, neither the Abstract nor the Summary of the Invention sections of the present document should be taken as containing a comprehensive listing of all such techniques or all aspects of such techniques.

It should be understood that the description and the drawings are not intended to limit the present techniques to the particular form disclosed, but to the contrary, the intention is to cover all modifications, equivalents, and alternatives falling within the spirit and scope of the present techniques as defined by the appended claims. Further modifications and alternative embodiments of various aspects of the techniques will be apparent to those skilled in the art in view of this description. Accordingly, this description and the drawings are to be construed as illustrative only and are for the purpose of teaching those skilled in the art the general manner of carrying out the present techniques. It is to be understood that the forms of the present techniques shown and described herein are to be taken as examples of embodiments. Elements and materials may be substituted for those illustrated and described herein, parts and processes may be reversed or omitted, and certain features of the present techniques may be utilized independently, all as would be apparent to one skilled in the art after having the benefit of this description of the present techniques. Changes may be made in the elements described herein without departing from the spirit and scope of the present techniques as described in the following claims. Headings used herein are for organizational purposes only and are not meant to be used to limit the scope of the description.

As used throughout this application, the word “may” is used in a permissive sense (i.e., meaning having the potential to), rather than the mandatory sense (i.e., meaning must). The words “include”, “including”, and “includes” and the like mean including, but not limited to. As used throughout this application, the singular forms “a,” “an,” and “the” include plural referents unless the content explicitly indicates otherwise. Thus, for example, reference to “an element” or “a element” includes a combination of two or more elements, notwithstanding use of other terms and phrases for one or more elements, such as “one or more.” The term “or” is, unless indicated otherwise, non-exclusive, i.e., encompassing both “and” and “or.” Terms describing conditional relationships, e.g., “in response to X, Y,” “upon X, Y,”, “if X, Y,” “when X, Y,” and the like, encompass causal relationships in which the antecedent is a necessary causal condition, the antecedent is a sufficient causal condition, or the antecedent is a contributory causal condition of the consequent, e.g., “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, e.g., the antecedent is relevant to the likelihood of the consequent occurring. Statements in which a plurality of attributes or functions are mapped to a plurality of objects (e.g., one or more processors performing steps A, B, C, and D) encompasses both all such attributes or functions being mapped to all such objects and subsets of the attributes or functions being mapped to subsets of the attributes or functions (e.g., both all processors each performing steps A-D, and a case in which processor 1 performs step A, processor 2 performs step B and part of step C, and processor 3 performs part of step C and step D), unless otherwise indicated. Similarly, reference to “a computer system” performing step A and “the computer system” performing step B can include the same computing device within the computer system performing both steps or different computing devices within the computer system performing steps A and B. Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection have some property should not be read to exclude cases where some otherwise identical or similar members of a larger collection do not have the property, i.e., each does not necessarily mean each and every. Limitations as to sequence of recited steps should not be read into the claims unless explicitly specified, e.g., with explicit language like “after performing X, performing Y,” in contrast to statements that might be improperly argued to imply sequence limitations, like “performing X on items, performing Y on the X′ed items,” used for purposes of making claims more readable rather than specifying sequence. Statements referring to “at least Z of A, B, and C,” and the like (e.g., “at least Z of A, B, or C”), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating,” “determining” or the like refer to actions or processes of a specific apparatus, such as a special purpose computer or a similar special purpose electronic processing/computing device. Features described with reference to geometric constructs, like “parallel,” “perpendicular/orthogonal,” “square”, “cylindrical,” and the like, should be construed as encompassing items that substantially embody the properties of the geometric construct, e.g., reference to “parallel” surfaces encompasses substantially parallel surfaces. The permitted range of deviation from Platonic ideals of these geometric constructs is to be determined with reference to ranges in the specification, and where such ranges are not stated, with reference to industry norms in the field of use, and where such ranges are not defined, with reference to industry norms in the field of manufacturing of the designated feature, and where such ranges are not defined, features substantially embodying a geometric construct should be construed to include those features within 15% of the defining attributes of that geometric construct. The terms “first”, “second”, “third,” “given” and so on, if used in the claims, are used to distinguish or otherwise identify, and not to show a sequential or numerical limitation. As is the case in ordinary usage in the field, data structures and formats described with reference to uses salient to a human need not be presented in a human-intelligible format to constitute the described data structure or format, e.g., text need not be rendered or even encoded in Unicode or ASCII to constitute text; images, maps, and data-visualizations need not be displayed or decoded to constitute images, maps, and data-visualizations, respectively; speech, music, and other audio need not be emitted through a speaker or decoded to constitute speech, music, or other audio, respectively. Computer implemented instructions, commands, and the like are not limited to executable code and can be implemented in the form of data that causes functionality to be invoked, e.g., in the form of arguments of a function or API call. To the extent bespoke noun phrases (and other coined terms) are used in the claims and lack a self-evident construction, the definition of such phrases may be recited in the claim itself, in which case, the use of such bespoke noun phrases should not be taken as invitation to impart additional limitations by looking to the specification or extrinsic evidence.

In this patent, to the extent any U.S. patents, U.S. patent applications, or other materials (e.g., articles) have been incorporated by reference, the text of such materials is only incorporated by reference to the extent that no conflict exists between such material and the statements and drawings set forth herein. In the event of such conflict, the text of the present document governs, and terms in this document should not be given a narrower reading in virtue of the way in which those terms are used in other materials incorporated by reference.

The present techniques will be better understood with reference to the following enumerated embodiments:

Embodiment 1. A tangible, non-transitory, machine-readable medium storing instructions that, when executed, effectuate operations comprising: accessing, with a computer system, clinical trial data of an ongoing clinical trial that is not yet complete, the clinical trial having a plurality of treatment groups and a plurality of patients in the treatment groups; detecting, with the computer system, with an anomaly detection model, an anomaly in the clinical trial data for a first patient, the anomaly corresponding to a first patient among the plurality of patients; in response to detecting the anomaly, with the computer system, classifying the first patient as anomalous in the trial; generating, with the computer system, by an artificial intelligence model, a candidate explanation for the detected anomaly by designating data in a record for the first patient as potentially correlated with the anomaly; and causing, with the computer system, the anomaly and the candidate explanation to be presented to a data safety monitoring board (“DSMB”) of the clinical trial.

Embodiment 2. The medium of embodiment 1, the operations comprising: algorithmically forming a plurality of different sub-populations of the plurality of patients to be evaluated for suitability or unsuitability of a treatment of the clinical trial before the clinical trial is complete.

Embodiment 3. The medium of embodiment 2, the operations comprising: determining, based on the clinical trial data, with the computer system, that the treatment of the clinical trial is suitable for a first sub-population among the plurality of sub-populations.

Embodiment 4. The medium of embodiment 2, the operations comprising: determining, based on the clinical trial data, with the computer system, that the treatment of the clinical trial is not suitable for a second sub-population among the plurality of sub-populations.

Embodiment 5. The medium of embodiment 2, comprising steps for detecting heterogeneous treatment effects.

Embodiment 6. The medium of embodiment 1, wherein: the anomaly detection model performs steps for detecting anomalies.

Embodiment 7. The medium of embodiment 1, the operations comprising: detecting, with the computer system, with another anomaly detection model, another anomaly in the clinical trial data for a second patient among the plurality of patients.

Embodiment 8. The medium of embodiment 1, the operations comprising: forming a patient profile of the first patient; and causing the patient profile to be presented to the DSMB.

Embodiment 9. The medium of embodiment 8, wherein forming a patient profile of the first patient comprises: generating a natural language text narrative of the first patient's history in the clinical trial with a language model; generating, with the language model or another language model, executable code configured to analyze the first patient's history in the clinical trial; and including a result of the analysis in the patient profile.

Embodiment 10. The medium of embodiment 1, the operations comprising: determining a potential cause of the anomaly by determining a value that correlates with the anomaly.

Embodiment 11. The medium of embodiment 1, the operations comprising: determining a potential cause of the anomaly with steps for determining a potential cause of an event.

Embodiment 12. The medium of embodiment 1, the operations comprising: receiving feedback from a first DSMB member on a scan of a printed document with unstructured hand drawn annotations; and inferring a structured representation of the unstructured hand drawn annotations.

Embodiment 13. The medium of embodiment 1, the operations comprising: receiving feedback from a second DSMB member with unstructured hand drawn annotations entered with a stylus on a tablet computer; and inferring a structured representation of the unstructured hand drawn annotations.

Embodiment 14. The medium of embodiment 1, the operations comprising: providing a portal on client computing devices of members of the DSMB through which DSMB members can view information about the clinical trial, provide comments, and vote.

Embodiment 15. The medium of embodiment 14, wherein the portal provides workflow management for the DSMB.

Embodiment 16. The medium of embodiment 1, the operations comprising: generating a plurality of visualizations of the clinical trial data; receiving a first set of hand-drawn, unstructured annotations on at least some of the visualizations from a first subset of members of the DSMB; receiving a second set of structured annotations typed into a text input associated with at least some of the visualizations from a second subset of members of the DSMB; and causing a user interface to be presented to the DSMB that includes both the first set of annotations in structured form and the second set of annotations.

Embodiment 17. The medium of embodiment 1, the operations comprising: obtaining and storing minutes of a periodic meeting of the DSMB.

Embodiment 18. The medium of embodiment 17, the operations comprising: generating the minutes with a language model based on audio or audio transcripts of the meeting of the DSMB formed with a speech-to-text model.

Embodiment 19. The medium of embodiment 1, the operations comprising: grouping the patients into subgroups with a clustering algorithm; generating profiles of each of at least some of the subgroups with a language model; and causing the profiles to be presented to the DSMB.

Embodiment 20. A method or computing system configured to execute the operations of embodiments 1-19.

What is claimed is:

1. A tangible, non-transitory, machine-readable medium storing instructions that, when executed, effectuate operations comprising:
accessing, with a computer system, clinical trial data of an ongoing clinical trial that is not yet complete, the clinical trial having a plurality of treatment groups and a plurality of patients in the treatment groups;
detecting, with the computer system, with an anomaly detection model, an anomaly in the clinical trial data for a first patient, the anomaly corresponding to a first patient among the plurality of patients;
in response to detecting the anomaly, with the computer system, classifying the first patient as anomalous in the trial;
generating, with the computer system, by an artificial intelligence model, a candidate explanation for the detected anomaly by designating data in a record for the first patient as potentially correlated with the anomaly; and
causing, with the computer system, the anomaly and the candidate explanation to be presented to a data safety monitoring board (“DSMB”) of the clinical trial.

2. The medium of claim 1, the operations comprising:
algorithmically forming a plurality of different sub-populations of the plurality of patients to be evaluated for suitability or unsuitability of a treatment of the clinical trial before the clinical trial is complete.

3. The medium of claim 2, the operations comprising:
determining, based on the clinical trial data, with the computer system, that the treatment of the clinical trial is suitable for a first sub-population among the plurality of sub-populations.

4. The medium of claim 2, the operations comprising:
determining, based on the clinical trial data, with the computer system, that the treatment of the clinical trial is not suitable for a second sub-population among the plurality of sub-populations.

5. The medium of claim 2, comprising steps for detecting heterogeneous treatment effects.

6. The medium of claim 1, wherein:
the anomaly detection model performs steps for detecting anomalies.

7. The medium of claim 1, the operations comprising:
detecting, with the computer system, with another anomaly detection model, another anomaly in the clinical trial data for a second patient among the plurality of patients.

8. The medium of claim 1, the operations comprising:
forming a patient profile of the first patient; and
causing the patient profile to be presented to the DSMB.

9. The medium of claim 8, wherein forming a patient profile of the first patient comprises:
generating a natural language text narrative of the first patient's history in the clinical trial with a language model;
generating, with the language model or another language model, executable code configured to analyze the first patient's history in the clinical trial; and
including a result of the analysis in the patient profile.

10. The medium of claim 1, the operations comprising:
determining a potential cause of the anomaly by determining a value that correlates with the anomaly.

11. The medium of claim 1, the operations comprising:
determining a potential cause of the anomaly with steps for determining a potential cause of an event.

12. The medium of claim 1, the operations comprising:
receiving feedback from a first DSMB member on a scan of a printed document with unstructured hand drawn annotations; and
inferring a structured representation of the unstructured hand drawn annotations.

13. The medium of claim 1, the operations comprising:
receiving feedback from a second DSMB member with unstructured hand drawn annotations entered with a stylus on a tablet computer; and
inferring a structured representation of the unstructured hand drawn annotations.

14. The medium of claim 1, the operations comprising:
providing a portal on client computing devices of members of the DSMB through which DSMB members can view information about the clinical trial, provide comments, and vote.

15. The medium of claim 14, wherein the portal provides workflow management for the DSMB.

16. The medium of claim 1, the operations comprising:
generating a plurality of visualizations of the clinical trial data;
receiving a first set of hand-drawn, unstructured annotations on at least some of the visualizations from a first subset of members of the DSMB;
receiving a second set of structured annotations typed into a text input associated with at least some of the visualizations from a second subset of members of the DSMB; and
causing a user interface to be presented to the DSMB that includes both the first set of annotations in structured form and the second set of annotations.

17. The medium of claim 1, the operations comprising:
obtaining and storing minutes of a periodic meeting of the DSMB.

18. The medium of claim 17, the operations comprising:
generating the minutes with a language model based on audio or audio transcripts of the meeting of the DSMB formed with a speech-to-text model.

19. The medium of claim 1, the operations comprising:
grouping the patients into subgroups with a clustering algorithm;
generating profiles of each of at least some of the subgroups with a language model; and
causing the profiles to be presented to the DSMB.

20. A method, comprising:
accessing, with a computer system, clinical trial data of an ongoing clinical trial that is not yet complete, the clinical trial having a plurality of treatment groups and a plurality of patients in the treatment groups;
detecting, with the computer system, with an anomaly detection model, an anomaly in the clinical trial data for a first patient, the anomaly corresponding to a first patient among the plurality of patients;
in response to detecting the anomaly, with the computer system, classifying the first patient as anomalous in the trial;
generating, with the computer system, by an artificial intelligence model, a candidate explanation for the detected anomaly by designating data in a record for the first patient as potentially correlated with the anomaly; and
causing, with the computer system, the anomaly and the candidate explanation to be presented to a data safety monitoring board (“DSMB”) of the clinical trial.
